(19) United States
(12) Patent Application Publication
(10) Pub. No.: US 2005/0165558 A1
(43) Pub. Date: Jul. 28, 2005

(54) Sequence detection system calculator

(76) Inventors: Robert G Becker, Wildwood, MI (US); Shawn D. Miller, Fenton, MI (US); Andrew T. Pinz, O Fallon, MI (US); Jeffrey V. Surry, Florissant, MI (US)

Correspondence Address: CAROL M. NIELSEN;WINSTEAD SECHREST & MINICK, P.C., 2400 BANK ONE CENTER, 910 TRAVIS STREET, HOUSTON, TX, 77002, US

(21) Appl. No.: 10/505,991

(22) PCT Filed: Feb. 26, 2003
(86) PCT No.: PCT/US03/05878

Related U.S. Application Data
(60) Provisional application No. 60/630,136, filed on Feb. 26, 2002.

(51) Int. Cl. G06F 19/00; G01N 33/48; G01N 33/50

(52) U.S. Cl. 702/20

(57) ABSTRACT

A computer-readable medium contains instructions for controlling a computer system in the analysis of an experiment to detect RNA or DNA in a sample by receiving exported cycle threshold values (exported C_{T} values) for a plate of wells from a polymerase chain reaction system and then calculates the delta C_{T}, the delta delta C_{T} and the relative transcriptional change for the sample. The results including the cycle threshold values inputted from the polymerase chain reaction system are then displayed.

### BACKGROUND

The polymerase chain reaction (“PCR”) has revolutionized nucleic acid research by providing a rapid means of amplifying specific nucleic acid sequences from complex genetic samples without the need for time-consuming cloning, screening and nucleic acid purification protocols. PCR was originally disclosed and claimed by Mullis et al. in U.S. Pat. Nos. 4,683,195, 4,683,202, and 4,965,188, hereby incorporated by reference. Since that time, considerable advances have been made in the reagents, equipment and techniques available for PCR. These advances have increased both the efficiency and utility of the PCR reaction, leading to its adoption to an increasing number of different scientific applications and situations.

The earliest PCR techniques were directed toward qualitative and preparative methods rather than quantitative methods. PCR was used to determine if a given sequence was present in any quantity at all or to obtain sufficient quantities of a specific nucleic acid sequence for further manipulation. Originally, PCR was not typically employed to measure the amount of a specific DNA or RNA present in a sample. Only in recent years has quantitative PCR come to the forefront of nucleic acid research.

PCR amplification of a specific segment of DNA, referred to as the template, requires that the nucleotide sequence of at least a portion of each end of the template be known. From the template, a pair of corresponding synthetic oligonucleotide primers (“primers”) can be designed. The primers are designed to anneal to the separate complementary strands of template, one on each side of the region to be amplified, oriented with its 3′ end toward the region between the primers. The PCR reaction has the DNA template along with a large excess of the two oligonucleotide primers and each deoxyribonucleoside triphosphate, a thermostable DNA polymerase and an appropriate reaction buffer. To effect amplification, the mixture is denatured by heat to cause the complementary strands of the DNA template to disassociate. The mixture is then cooled to a lower temperature to allow the oligonucleotide primers to anneal to the appropriate sequences on the separated strands of the template. Following annealing, the temperature of the reaction is adjusted to an efficient temperature for 5′ to 3′ DNA polymerase extension of each primer into the sequences present between the two primers. This results in the formation of a new pair of complementary strands. The steps of denaturation, primer annealing and polymerase extension can be repeated many times to obtain a high concentration of the amplified target sequence. Each series of denaturation, annealing and extension constitutes one “cycle.” There may be numerous “cycles.” The length of the amplified segment is determined by the relative positions of the primers with respect to each other, and therefore, this length is a controllable parameter. By virtue of the repeating aspect of the process, the method is referred to as the “polymerase chain reaction” (hereinafter “PCR”).

As the desired amplified target sequence becomes the predominant sequence in terms of concentration in the mixture, this sequence is said to be PCR amplified. With PCR, it is possible to amplify a single copy of a specific target sequence in genomic DNA to a level detectable by several different methodologies. These methodologies include ethidium bromide staining, hybridization with a labeled probe, incorporation of biotinylated primers followed by avidin-enzyme conjugate detection, and incorporation of ³²P-labeled deoxynucleotide triphosphates such as dCTP or DATP into the amplified segment. In addition to genomic DNA, any oligonucleotide sequence can be amplified with the appropriate set of primer molecules. In particular, the amplified segments created by the PCR process are efficient templates for subsequent PCR amplifications leading to a cascade of further amplification. Furthermore, amplification of RNA into DNA can be accomplished by including a reverse transcription step prior to the start of PCR amplification.

Prior to the development of real-time PCR, hybridization techniques were most commonly used for the quantification of specific nucleic acids. The hybridization signals in the test sample would be compared to similar signals in serial dilutions of samples of known concentration. However, hybridization can be a time consuming process and requires large amounts of starting material.

While the potential application of PCR to the quantification of nucleic acid sequences was recognized almost immediately following its development, numerous technical difficulties delayed the acceptance of quantitative PCR as a reliable technique. Theoretically, each strand of template DNA should be copied during PCR amplification, resulting in the exponential amplification of the target sequence. In practice, however, not every template is copied during each cycle.

Other technical difficulties such as the presence of competing templates or the presence of inhibitors in the template sample can delay the exponential phase of the amplification for several cycles. In later cycles, the rate of DNA amplification begins to plateau as the deoxyribonucleoside triphosphates and primers are incorporated into the template and become limited in concentration. As a result, quantification of product is most reliable if measured during the exponential phase of DNA amplification. However, because of variations in the quantity and quality of the DNA template and in the efficiency of annealing between different sequences, it is difficult to predict the timing and duration of the exponential phase of amplification.

Early attempts to achieve verifiably quantitative PCR involved the creation of standardized curves by stopping the reaction at various points and removing aliquots from the reaction. In this manner, the rate of amplification could be plotted to identify the exponential phase of amplification. However, detection of product in the early stages of amplification required radioactive labeling with all of its inherent technical difficulties and hazards. In addition, multiple dilutions of the template and multiple samplings were often necessary to obtain a linear standard curve, resulting in the need for multiple reactions. As a result, these methods were costly in terms of template and reagent as well as tedious to perform. Competitive PCR was developed in an attempt to solve these problems.

In competitive PCR, two templates are included in each PCR reaction, a control template of known concentration and a test template of unknown concentration. The control template may have nearly the same sequence as the test template but varies enough to be independently detectable. It may differ in size or may have point mutations or restriction sites not present in the test template. After the PCR reaction is completed, the product yields are measured for each template and the amount of test template is calculated from the known concentration of the control template. This method, while a considerable improvement, still suffers from a number of limitations. Even though the differences between the control template and test template are minor, these may still be enough to alter the rate of amplification. However, at the same time, the control template and test template may be similar enough that the individual strands of the test and control products may associate with each other to form heterodimers. In addition, competitive PCR works best if the test and control DNA are present in nearly equal amounts. Thus, multiple dilutions are often still necessary with all the accompanying increased costs in terms of labor, reagents and starting materials.

The development of real-time PCR, also known as kinetic PCR, has provided an improved method for the quantification of specific nucleic acids. In real-time PCR, cycle-by-cycle measurement of accumulated PCR product is made possible by combining thermal cycling and fluorescence detection of the amplified product in a single instrument. Because the product is measured at each cycle, product accumulation can be plotted as a function of cycle number. The exponential phase of product amplification is readily determined and used to calculate the amount of template present in the original sample. A number of alternative methods are currently available for real-time PCR

The original protocol developed by Grossman et al. (U.S. Pat. No. 5,470,705, hereby incorporated by reference) used radioactive labels on the probes but further refinements of the method have focused on self-quenching fluorescent probes. Originally, separation of the amplified products by electrophoresis or other methods was used to measure and calculate the amount of released label. This added time-consuming steps to the analysis. Furthermore, this end-stage analysis of the reactions cannot be readily applied to real-time PCR.

In one current method, fluorogenic exonuclease probes for the real-time detection of PCR products are used. This type of technology is captured in the ABI Prism® 7700 Sequence Detection System and disclosed in Livak et al (U.S. Pat. No. 5,538,848 hereby incorporated by reference). In a modification of an existing method utilizing radioactive labels, fluorogenic exonuclease probes are designed to anneal to sequences between the two amplification primers but contain one or more nucleotides that do not match at the 5′ end. The nonmatching nucleotides are linked to a fluorescence donor. A fluorescence quencher is positioned typically at the end of the probe. When the donor and quencher are in the same vicinity, the quencher prevents the fluorescence donor from emitting light.

Traditional fluorescence quenchers absorb light energy emitted by an excited reporter molecule and release this energy by fluorescing at a higher wavelength. Increased sensitivity in real-time detection can be achieved with dark quenchers such as dabcyl or the developed Eclipse Quencher from Epoch Biosciences, Inc. The dark quenchers absorb fluorescent energy but do not fluoresce themselves, thus reducing background fluorescence in the sample. The dark quencher works effectively against a number of red-shifted fluoropores such as FAM, Cy3 and Tamra due to its broader range of absorbance over dabcyl (400-650 nm versus 360-500 nm respectively) and is thus better suited to multiplex assays.

The sensitivity of real-time PCR can also be augmented through the use of minor groove binders (“MGBs”) (also from Epoch Biosciences, Inc.), which are certain naturally occurring antibiotics and synthetic compounds able to fit into the minor groove of double-stranded DNA to stabilize DNA duplexes. The minor groove binders can be attached to the 5′ end, 3′ end or an internal nucleotide of oligonucleotides to increase the oligonucleotide's temperature of melting, i.e., the temperature at which the oligonucleotide disassociates from its target sequence and hence creates stability. The use of MGBs allows for the use of shorter oligonucleotide probes as well as the placement of probes in AT-rich sequences without any loss in oligonucleotidal specificity, as well as better mismatch discrimination among closely related sequences. Minor group binders may be used in connection with dark quenchers or alone.

Thermus aquaticus (taq) DNA polymerse used for the PCR amplification has the ability to cleave unpaired nucleotides off of the 5′ end of DNA fragments. In the PCR reaction, the fluorogenic probe anneals to the template (the nucleotide sequence of interest in a sample). An extension of both primers and the probe occurs until one of the amplification primers is extended to the probe. Taq polymerase then cleaves the nonpaired nucleotides from the 5′ end of the probe, thereby releasing the fluorescence donor. Once it is physically separated from the quencher, the fluorescent donor can fluorescence in response to light stimulation. Because of the role of taq polymerase in this process, these probes are often referred to as TaqMan® probes. As more PCR product is formed, more fluorescent donors are released, allowing the formation of the PCR product to be measured and plotted as a function of cycle time. The linear, exponential phase of the plot can be selected and used to calculate the amount of nucleotide in the sample. The development of these self-quenching fluorescent probes was a considerable advancement in quantitative PCR. Numerous improved self-quenching probes and methods for the use thereof have been subsequently reported in U.S. Pat. Nos. 5,912,148, 6,054,266 (Kronick et al.) and U.S. Pat. No. 6,130,073 (Eggerding).

The LightCycler® uses hybridization instead of exonuclease cleavage to quantitate the amplification reaction. This method also adds additional fluorogenic probes to the PCR amplification. However, unlike the TaqMan® system, fluorescence increases in this system when two different fluorogenic probes are brought together on the same template by extension or hybridization, allowing resonance energy transfer to occur between the two probes.

Other systems are also available. The Amplifluor® primers produced by Intergen® are hairpin oligonucleotides, which form hairpins when they are single, stranded, which bring a fluorescence donor and quencher into close proximity. When the primers are incorporated into a double stranded molecule, the hairpins are straightened, which separates the donor and quencher to cause an increase in fluorescence.

Other applications make use of intercalating dyes, which only associate with double stranded DNA. As more double stranded DNA is generated by the reaction, more fluorescence is observed as more dye becomes associated with DNA.

Regardless of the method used, the end result is the same, a plot of fluorescence versus cycle number. Further analysis of this data is then used to derive quantitative values for the RNA's present in the samples. Successful amplification of the sample will result in a sigmoidal plot consisting of a period where amplification is not detectable above the background noise of the experiment, a period of exponential amplification and a period where amplification plateaus. To analyze the data, threshold value is selected that is greater than the background noise of the experiment. Each amplification curve is analyzed to determine the point at which the curve rises above the threshold values. This is recorded in terms of the cycle in which this occurred and is known as the threshold cycle (C_{T}).

As originally published in User Bulletin #2 for ABI Prisim 7700 Sequence Detection System, incorporated herein by reference, in the linear range (or exponential phase) the threshold cycle is inversely proportional to the amount of RNA in a sample. These values can be compared to a plot of threshold cycles obtained from amplification of serial dilutions of an exogenously added standard to determine the concentration RNA in the experimental samples. If the absolute quantity of the exogenously added standard is known, the absolute quantities of RNA in the experimental samples can be determined. However, the standard can also be of unknown concentration, in which case, relative quantitation will be obtained.

The use of standard curves requires the amplification of exogenously added nucleic acids, increasing the total number of amplifications required and lowering the throughput of the experiment. Furthermore, because of variations in the quantity and quality of nucleic acids between different samples, it is often beneficial to compare the amount of nucleic acid to an endogenous control. If an endogenous control is present, relative quantitation can be accomplished by mathematical analysis of the differences in cycle threshold between the experimental sample and the endogenous control, eliminating the need for standard curves and reducing the total number of amplification required in an experiment. This mathematical analysis is performed by the human investigator and can take weeks to prepare, publish and analyze. Au automated way of preparing the data for analysis to meet the high-throughput requirements of today's drug discovery process is lacking.

A need exists therefore, for an effective and efficient way of analyzing the results of a high through put experiment to detect specific DNA or RNA transcripts.

### SUMMARY OF THE INVENTION

The subject invention is a method in a computer system for analyzing an experiment to detect RNA or DNA from a two dimensional plate configuration. The method comprises the steps of: (1) recording experiment information; (2) specifying at least one plate to the experiment, each plate having a series of wells and dye layers and at least one forward primer, probe, and reverse primer set (“FPR set”) categorized by dye layer or well; (3) populating at least one RNA group; (4) receiving exported experimental cycling results for each plate including a cycle threshold value (“C_{T}”) value for each FPR set in each well; (5) calculating delta C_{T}, delta delta C_{T}, and relative transcriptional change (XRel) values for each sample RNA; and (6) displaying the C_{T}, the delta C_{T}, the delta delta C_{T}, and the XRel values for each sample RNA to detect RNA.

A computer-readable medium contains instructions for controlling a computer system in the analysis of an experiment to detect RNA or DNA in a sample by receiving exported cycle threshold values (exported C_{T} values) for a plate of wells from a sequence detection system (also referred to herein as “a polymerase chain reaction system”) and then calculating the delta C_{T}, the delta delta C_{T} and the relative transcriptional change for the sample. The results including the cycle threshold values inputted from the polymerase chain reaction system are then published/displayed.

The present invention includes a computer readable program embodied in a computer readable medium for analyzing data from any two dimensional plate configurations, such as 96-well plates, 96-well custom plates, 384-well plates and 384-well custom cards. The computer program of the subject invention may also be used with an information-display apparatus. The program rapidly calculates the delta C_{T}, delta delta C_{T} and XRel values for each dye layer of each well of a plate saving weeks of time in the analysis.

In the method of the present invention, any plate layout, unlimited numbers of RNAs and unlimited numbers of primer/probe sets in an experiment are acceptable. Data may be analyzed from a partial plate, single plate or multi-plate experiment. Single dye or multiplex analysis (not limited to two dyes) may be accommodated.

Exported results from any assay may be used for calculation. Output may be published in an Excel spreadsheet format and the like or on information-display apparatus. The output includes the delta C_{T}, the delta delta C_{T}, and the relative change in transcription or relative expression values (XRel). The method of this invention provides the flexibility to choose which FPR set is treated as the endogenous control when multiplexing, and which RNA group is treated as the comparator group, making it possible to compare reports with different endogenous control/comparator group combinations. In addition, the % CV between replicate wells on a plate is calculated and outlier replicates are flagged. The method of the subject invention may also be used to generate the mean, standard deviation, and standard error of the mean among RNA groups.

Experiment analysis using the method of the subject invention involves a series of steps. Each step includes specifying certain information in order to create file formats that receive exported cycle threshold values for a given plate of wells, later used to calculate the delta C_{T}, the delta delta C_{T} and the relative transcription change (XRel).

As further described below, in the first step, experiment information is defined such as a description, number of dye layers and other parameters. Plate size, real or virtual plates and standard or custom cards is then specified as plate information. The plate layout including drawing the plates or copying the layout of existing plates is added to the plate information. FPR sets, RNA, and well type may also be part of the plate layout information provided. RNA is assigned to a group as group information. An endogenous control may be selected and the file information saved. Raw data from the PCR system is then viewed and outliers set. The delta C_{T}, the delta delta C_{T} and the relative transcription change are calculated, and these values are then published.

### BRIEF DESCRIPTION OF SEVERAL VIEWS OF THE DRAWINGS

For better understanding of the invention and to show by way of example how the invention may be carried into effect, reference is now made to the detail description of the invention along with the accompanying figures in which corresponding numerals in the different figures refer to corresponding parts and in which:

FIG. 1 is a logic flow diagram depicting the overall methodology of the present invention.

FIG. 2 is a logic flow diagram depicting step 1, experiment information.

FIG. 3 is a logic flow diagram depicting step 2, plate information.

FIG. 4 is a logic flow diagram depicting step 3, plate layout.

FIG. 5 is a logic flow diagram depicting step 4, group information.

FIG. 6 is a logic flow diagram depicting step 5, file information.

FIG. 7 is a logic flow diagram depicting step 6, raw data and outlier management.

FIG. 8 is a logic flow diagram depicting step 7, calculation.

FIG. 9 is a logic flow diagram depicting step 8, publish.

### DETAILED DESCRIPTION

The present invention is a method in a computer system for analyzing an experiment to detect RNA or DNA from a two-dimensional plate configuration. A computer-readable medium contains instructions for controlling a computer system in the analysis of an experiment to detect RNA in a sample. The computer usable medium has a computer readable program code embodied therein for determining the presence of RNA in a sample contained within a dye layer of a well of a plate. A program storage device readable by a computer, tangibly embodies the program of instructions is executed by the computer and performs the method steps for analyzing the presence of RNA in a sample. Also provided is a computer-readable medium containing a data structure. A memory for storing data for access by the computer program comprises the data structure.

The present invention is suitable for any two-dimensional plate configuration including but not limited to 96-well plates, 384-well plates, custom or standardized. The invention has the capability to analyze data from a partial plate, single plate or multi-plate experiment. Single dye or multiple dye (multiplexed) analysis can also be accommodated. The computer readable program code will accept any plate layout, unlimited number of RNA samples and unlimited number of primer/probe sets (FPR sets) in an experiment. Exported result files from any experiment run can be loaded into the program for calculation.

As part of the subject invention, the user may choose which FPR set is treated as the endogenous control and which RNA group is treated as the comparator group, making it possible to compare reports with different endogenous control and comparator group combinations. In addition, percent CV (% CV) between replicate wells on a plate may be calculated and outlier replicates are flagged. The mean, standard deviation, and standard error of the mean among RNA groups may also be calculated.

As further described below, experiment analysis involves a series of steps. The results of the analysis may be displayed in a Microsoft excel workbook and the like or on an information-display apparatus.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

As used throughout the present specification the following abbreviations are used:
C_{T} means threshold cycle value and is the cycle during PCR when there is a detectable increase in signal intensity or fluorescence above baseline;
CV means the coefficient of variation that is calculated for each set of replicate wells having the same group label, sample ID, and gene;
ΔC_{T} (also referred to as “delta C_{T}”)=Mean (C_{T} values for sample FPR)−Mean(C_{T} values Endogenous Control FPR)
ΔC_{T} Mean Vehicle (comparator group)=Mean(ΔC_{T} for all amplifications of the FPR set in the comparator group)
ΔC_{T} Median Vehicle (comparator group)=Median(ΔC_{T} for all amplifications of the FPR set in the comparator group)
ΔΔC_{T} (also referred to as “delta delta C_{T}”)=(ΔC_{T} for the sample, treated or diseased)−ΔC_{T} Median Vehicle(comparator group)
E means to the efficiency of amplification for each experiment and is assumed to be 1 (one);
FPR set means Forward Primer, Probe, and Reverse Primer Set used to identify the presence of a gene;
−RT means Minus Reverse Transcriptase, an amplification used to determine if DNA contaminants exist in the RNA. A −RT well contains RNA and an FPR set, but does not contain reverse transcriptase. Minus reverse transcriptase wells are related to sample wells that have the same RNA and FPR set as the −RT well.
NTC means no template control and is a well that contains no RNA;
PCR means polymerase chain reaction;
Rₙ, normalized reporter signal and is determined to be the signal activity of the reporter dye divided by the signal activity of the passive reference dye;
RT means reverse transcriptase;
XRel means relative transcriptional change or relative expression level of the gene.

Additional terms as used through the specification are defined as follows:

Amplify when used in reference to nucleic acids refers to the production of a large number of copies of a nucleic acid sequence by any method known in the art. Amplification is a special case of nucleic acid replication involving template specificity.

Comparator or Comparator Group refers to sample used as the basis for comparative results.

Dye refers to any fluorescent or non-fluorescent molecule that emits a signal upon exposure to light as apparent to those of skill in the art of molecular biology. The reporter dye refers to the dye used with the sample RNA.

Endogenous control refers to an RNA or DNA that is always present in each experimental sample. By using an endogenous messenger RNA (mRNA) target can be normalized for differences in the amount of total RNA added to each reaction. Typically, the endogenous control is a housekeeping gene required for cell maintenance such as a gene for metabolic enzyme or the ribosomal RNA.

Exogenous control refers to a characterized RNA or DNA spiked into each sample at a known concentration. An exogenous active reference is usually an in vitro construct that can be used as an internal positive control (IPC) to distinguish true target negatives from PCR inhibition. An exogenous reference can also be used to normalize for differences in efficiency of sample extraction or complementary DNA (cDNA) synthesis by reverse transcriptase.

Experiment means a group of plates analyzed together;

Gene is used to refer to a functional protein, polypeptide or peptide-encoding unit. As will be understood by those in the art, this functional term includes genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

Multiplexing PCR means the use of more than one dye layer in an experiment and/or more than one FPR set with an associated reporter dye in each well of a plate. In one well, the target RNA and the endogenous control are amplified by different FPR sets. All the wells on a plate in an experiment will always contain the same endogenous FPR set. If there are three FPR sets used in the experiment, then all wells will have at least one of those same three FPR sets unless the wells are empty wells on the plate. Each FPR set has an associated reporter dye. A C_{T} value is reported for each FPR set in each well. A C_{T} value is recorded for each dye layer in every well on the plate.

Notebook Page means a page in a notebook used to track experiments and other confidential information.

Nucleic acid refers to DNA, RNA, single-stranded or double-stranded and any chemical modifications thereof. Modifications include, but are not limited to, those that add other chemical groups that provide additional charge, polarizability, hydrogen bonding, and electrostatic interaction.

Plate Consistency Control means a specified RNA, which is placed on every plate in multiple plate experiments to ensure consistency across plates. Primer refers to an oligonucleotide, whether purified or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer may be single stranded for maximum efficiency in amplification but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, source of primer and the use of the method.

Probe refers to any compound which can act upon a nucleic acid in a predetermined desirable manner, including a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell. It also refers to a sequence of nucleotides, whether purified or produced synthetically, recombinantly or by PCR amplification, which is capable of hybridizing to another nucleotide sequence of interest. A probe may be single-stranded or double-stranded. Probes are useful in the detection, identification and isolation of particular gene sequences. It is contemplated that any probe used in the present invention will be labeled with a “reporter molecule,” so that is detectable in any detection system including, but not limited to, enzyme (e.g. ELISA, as well as enzyme-based histochemical assays), fluorescent, radioactive, and luminescent systems. It is not intended that the present invention be limited to any particular detection system or label.

Reference refers to a passive or active signal used to normalize experimental results. Endogenous and exogenous controls are examples of active references. Active reference means the signal is generated as a result of PCR amplification. The active reference has its own set of primers and probe.

Sample RNA or sample refers to single or double stranded RNA used in one or more experiments that may be obtained from a donor such as a person, animal or cell culture. When from an animal or person, it may be from variety of different sources, including blood, plasma, urine, semen, saliva, lymph fluid, meningeal fluid, amniotic fluid, glandular fluid, and cerebrospinal fluid, or from solutions or mixtures containing homogenized solid material, such as feces, cells, tissues, and biopsy samples. One RNA sample may be used to determine the expression of one or more genes. The same set of genes is used with every sample in the same experiment.

Standard refers to a sample of known concentration used to construct a standard curve.

Vehicle refers to substances that are injected into an animal as carriers for a test compound. Common vehicles include water, saline solutions, physiologically compatible organic compounds such as various alcohols, and other carriers well known in the art. Vehicle may also refer to a control animal injected with such a carrier in the absence of a test compound. The vehicle animal serves as a control to mimic transcriptional alterations resulting from the stress of administration but not from the drug itself.

### Calculations

As further described below in detail, the following calculations are used in connection with the subject invention:
% CV for C_{T} Values(Coefficient of Variation)=100*(StDev/Mean)

XRel (relative transcriptional change or relative expression level), This value is calculated as (1+E)(^{-ΔΔC}_{T} for FPR set), where E reflects the amplification efficiency and is assumed to be 1. E is stored as an experiment parameter and can be changed if necessary for the given experiment. XRel values greater than 1 (one) indicate more gene expression in the RNA sample than in the comparator group of the particular gene. Similarly, XRel values less than 1 (one) indicate less gene expression in the RNA sample than in the comparator group of the particular gene.
Group XRel Mean=Mean (XRel of each amplification of the FPR set in the group
Group XRel StDev=StDev(XRel of each amplification of the FPR set in the group
Group XRel SEM=StDev(XRel of each amplification of the FPR set in the group/(n)⁵, where n is the number of amplifications with FPR set in the group
% CV XREL=100*XRel SEM*SQR_{T}(n)/XRel Mean, where n is the number of RNAs in the group.

If the amplification primers are optimized for amplification efficiency (i.e. E=1), XRel, the amount of a nucleic acid sample normalized to an endogenous reference and relative to a comparator group can be calculated by the mathematical formula:
XRel=2^{-ΔΔCT}

This above formula was derived in the following manner: The exponential amplification resulting from a given PCR reaction can be represented by the formula:
Xₙ=Xₒ×(1+E_{X})ⁿ
where Xₙ is the number of sample molecules after n cycles, X₀ is the initial number of sample molecules; Eₓ is the efficiency of sample amplification; and, n is the number of cycles.

This formula is then used to calculate the amount of product present at the threshold cycle, C_{T}. The threshold cycle is the point at which the amount of sample rises above a set threshold, typically where exponential amplification can be first detected above the background noise of the experiment. At this point, the amount of product is:
X_{T}=Xₒ×(1+E_{X})^{C}^{_{T,X}}=K_{X}
where X_{T} is the number of sample molecules at the threshold cycle, C_{T,X} is the cycle number at which the amount of sample exceeds the threshold value, and Kₓ is a constant.

In addition, a similar formula can be used to calculate the amount of amplified sample in the endogenous reference control reaction at its threshold cycle:
R_{T}=Rₒ×(1+E_{R})^{C}^{_{T,R}}=K_{R}
where R_{T} is the number of copies of the amplified endogenous reference at its threshold cycle, R₀ is the initial number of copies of the endogenous reference, E_{R} is the efficiency of amplification of the endogenous reference, C_{T,R} is the threshold cycle number for the endogenous reference, where the amplified reference exceeds the threshold value, and K_{R} is a constant for the endogenous reference.

The number of sample molecules (X_{T}) at the sample threshold cycle is then divided by the number of endogenous reference molecules at the reference threshold cycle to yield a constant designated as K:

$$\frac{{X}_{T}}{{R}_{T}} = \frac{{X}_{o} \times {( 1 + {E}_{X} )}^{{C}_{T , X}}}{{R}_{o} \times {( 1 + {E}_{R} )}^{{C}_{T , R}}} = \frac{{K}_{X}}{{K}_{R}} = K$$

The constant, K, is not necessarily equal to one because the exact values of X_{T} and R_{T} can vary for a number of reasons depending on the reporter dyes used in the probes, differential effects of probe sequences on the fluorescence of the probes, the efficiency of probe cleavage, the purity of the probes, and the setting of the fluorescence threshold.

If the amplification efficiencies of the sample and endogenous reference are assumed to be the same, i.e. E_{X}=E_{R}=E, the previous equation can be simplified to:

$$\frac{{X}_{o}}{{R}_{o}} \times {( 1 + E )}^{{C}_{T , X} - {C}_{T , R}} = K$$

which can be rewritten as:
X_{N}×(1+E)^{ΔC}^{_{T}}=K
where X_{N} is the normalized amount of sample (X₀/R₀); and ΔC_{T} is the difference in threshold cycles for the sample and reference (C_{T,X}-C_{T,R}). The equation can be rearranged as follows:
X_{N}=K×(1+E)^{-ΔC}^{_{T}}

XRel is then obtained by dividing normalized amount of sample relative to endogenous control by the normalized amount of comparator relative to endogenous control as represented by the equation:

$$\frac{{X}_{N , q}}{{X}_{N , cb}} = \frac{K \times {( 1 + E )}^{- Δ {C}_{T , q}}}{K \times {( 1 + E )}^{- Δ {C}_{T , cb}}} = {( 1 + E )}^{- ΔΔ {C}_{T}}$$

where the ΔΔC_{T}=ΔC_{T,q}−ΔC_{T,cb}. If the FPR sets are properly optimized for amplification efficiency, E should be nearly equal to one and the equation can be simplified to:
XRel=2^{-ΔΔC}^{_{T}}

For a given experiment, sample RNA may be obtained from a variety of sources. It may be animal tissue from a particular organ or animal blood or it might be from cell cultures. Regardless, there is usually more than one sample having the same characteristic. The common characteristic may be the type of treatment received (vehicle, compounds, etc.), the species, sex or age of the donor, or some other similar treatment. A group label is assigned to each group of samples sharing the same characteristic. Cell culture experiments in which each well on the cell culture plate is treated differently and not replicated on another cell culture plate, will result in only one sample per group. Statistical analysis assumes there is more than one sample per group and that each sample is independent of other samples treated in the same manner.

One of the groups must be identified as the comparator group. It is often the vehicle or untreated group. The comparator group may also be a particular age or time point in the experiment. The comparator group is the one group which all other groups in that experiment will be compared. For example, the comparator group may be untreated or normal sample to which the treated or diseased samples are compared. All relative expression values are defined relative to the comparator group as being either the same, higher or lower than the comparator group.

Occasionally in an experiment, there may be a need to calculate relative expression values several times using more than one comparator group. For example, it may be necessary to see the relative fold changes in a message compared to different time points in an experiment thus creating the need to easily be able to change the comparator group and quickly recalculate the relative expression values.

To run the experiment to detect RNA in the sample, the current technology of either 96 or 384 well plates may be used. C_{T} values of each well are typically supplied by the manufacturer of the polymerase chain reaction system (otherwise referred to herein as the sequence detection system). Each well may be identified as containing sample RNA or one of several types of assay controls.

There may be one or more types of control wells on each plate, or there may be no control wells. The most common type of control occurs when the experiment is performed on more than one plate, and will therefore be called the plate control. The plate controls have the same source of RNA on all the plates and are monitored to determine whether there is consistency in results across plates. The plate control may be one of the samples for which there is sufficient RNA to repeat it on all of the plates. Another type of control well is called the no template control, or NTC where no RNA is present. Thus, this control is used to determine the background signal. A third type of control well is called minus reverse transcriptase control, or −RT. These wells contain no reverse transcriptase. Thus, this control is used to check whether DNA contaminants are present in the RNA preparation.

If there are multiple plates and custom cards are not used, there should be plate controls on each plate. These RNA controls are usually matched to each gene (including endogenous) by being in the same rows or same columns as the RNA samples for that gene.

All samples and controls are replicated, having two or more wells for each sample or control. The replicates must be on the same plate and will usually be in the same row or the same column.

The RNA samples may be tested for the expression of one or more genes. The same set of genes is used with every sample in the same experiment. There will be matching endogenous control wells for each set of gene wells on the same plate that will be used in the calculations. The most common endogenous control is cyclophilin. These endogenous controls will usually be in the same rows or the same columns as the gene sample. If a sample is run for multiple genes on the same plate, the same endogenous control is used for all genes. If more than one endogenous control is present, only one will be identified for use in the calculations.

An exception occurs when custom plates are used. For example, one RNA sample may be analyzed for the transcription levels of genes plus one endogenous control. Having the endogenous control contained in the same well as the gene is called multiplexing.

Preferably, each well is specified by the following information:
1) well location on the plate or custom card,
2) sample type (unused, assay control, RNA sample, or both assay control and RNA sample),
3) group label (such as treatment group, species, sex, age, type of control, etc.),
4) sample ID (usually a number) within the group,
5) number of the FPR set(s) that identifies the gene(s).

If the sample type is assay control, the group label will identify the type of control as either plate RNA control, NTC, or −RT. The sample id field may be used to indicate the particular RNA sample corresponding to the control. For example, each RNA (sample ID) may have a −RT corresponding to it to check for DNA contamination in that sample preparation. The FPR set number identifies the gene label for the plate RNA, NTC, or −RT control results and graphs.

For the RNA samples, the sample ID may be an RNA ID from remote database or an assigned name or number. There may be two FPR set numbers for the same well, one for the endogenous control and one for the gene, if multiplexing is being performed, as in custom cards. Multiplexing can be done on regular samples or on custom plates but is not necessarily done on either.

If statistical comparisons are going to be made among groups, whenever possible, it is desirable to have the samples for the various groups on the same plate. However, it is understood that this type of plate setup is not always possible. The coefficient of variation (100× standard deviation/mean), or CV, may be calculated for each set of replicate wells having the same group label, sample ID, and gene. The well locations of sets of wells where the CV exceeds a default value (currently 2% but may be lower), or a value that is specified by the user, are shown. The user may than choose whether to delete one or more of these wells from further processing.

The average of the replicate values is calculated for each assay control, endogenous control, and gene. A ΔC_{T} (delta C_{T}) value is calculated for each RNA sample/gene combination as the average C_{T} for the gene minus the average C_{T} for the endogenous control for that sample.

The calculations described so far can be performed at the plate level. The rest of the calculations require the data for all the plates to be available. All of the samples for the comparator group may not be on the same plate. Also, the samples for the comparator group may or may not be on the same plate as the samples for the other groups.

The median of the C_{T} values is determined for all the samples in this comparator group, regardless of plate location. Then a ΔΔ(delta delta C_{T}) value is calculated as the ΔC_{T} value for each sample minus the median (middle or average of two middle values) ΔC_{T} value for the comparator group.

As mentioned above, the relative transcriptional change (or relative expression level), XRel, is calculated as (1+E)^{(-ΔΔCT)}. E reflects the amplification efficiency and defaults to 1. Since ΔΔC_{T} will be about zero for the comparator group, its XRel value will be close to 1. XRel values greater than 1 indicate more expression than the comparator group while XRel values less than 1 indicate less expression than the comparator group by the particular gene.

There are special rules for multiplexing. When multiplexing, any given FPR set cannot exist in more than one combination of FPR sets. For example, if Gene 2 exists in a well with Gene 1 and the endogenous control (“EndoC_{T}”), then Gene 2 can ONLY exist in wells also containing Gene 1 and the endogenous control (“EndoC_{T}”). Gene 2 cannot exist in a well of any other combination. For example, Gene 2 cannot exist in a well containing Gene 3 and the EndoC_{T}. Any multiplexing experiment not following this rule will result in the reporting of invalid calculations. Below is an example of two plates. Plate 1 is a valid multiplexing experiment, while Plate 2 is not a valid multiplexing experiment.

| | | | |
|---|---|---|---|
| Endo, Gene1, Gene2 | Endo, Gene1, Gene2 | Endo, Gene3, Gene4 | Endo, Gene3, Gene4 |

PLATE 1: Multiplexing (valid case)

| | | | | |
|---|---|---|---|---|
| RNA1 | CTe, CTg1, CTg2 | CTe, CTg1, CTg2 | CTe, CTg3, CTg4 | CTe, CTg3, CTg4 |
| RNA2 | CTe, CTg1, CTg2 | CTe, CTg1, CTg2 | CTe, CTg3, CTg4 | CTe, CTg3, CTg4 |
| RNA3 | CTe, CTg1, CTg2 | CTe, CTg1, CTg2 | CTe, CTg3, CTg4 | CTe, CTg3, CTg4 |

PLATE 2: Multiplexing (invalid case - Gene2 is a
part of more than one FPR set combination)

| | | | | |
|---|---|---|---|---|
| RNA1 | CTe, CTg1, CTg2 | CTe, CTg1, CTg2 | CTe, CTg2, CTg3 | CTe, CTg2, CTg3 |
| RNA2 | CTe, CTg1, CTg2 | CTe, CTg1, CTg2 | CTe, CTg2, CTg3 | CTe, CTg2, CTg3 |
| RNA3 | CTe, CTg1, CTg2 | CTe, CTg1, CTg2 | CTe, CTg2, CTg3 | CTe, CTg2, CTg3 |

There are various levels of documentation and display of information. From the PCR system, typically a printout or other display is obtained that shows the details of the C_{T} values for each well on the plate. The present invention calculates and displays from these values should show, by gene, the C_{T}, ΔC_{T}, ΔΔC_{T}, and XRel values for each sample in each group.

In addition, a summary may be shown for each group and gene that contains the descriptive statistics for the group (n, mean, and standard error of the mean). A graph may be produced for each gene that displays the group means (with error bars). Furthermore, an electronic output file should be generated that contains the XRel values for each sample along with the gene label, group label, and sample id. This output file can then be used for further statistical analysis.

More detailed database files may be produced using the original plate reader values so that, if desired, the calculations may be re-done, exercising different options. Graphics may be produced for assay validation purposes. Assuming that the C_{T} values are available on the same plate for endogenous control samples and assay controls, a graph is produced whose X-axis may display the C_{T} average of the endogenous control wells that were used to calculate ΔC_{T} for all of the genes on the plate. The Y-axis may then display the C_{T} values for the various types of assay control wells, by gene, including endogenous. The symbol printed reflects the gene label, as described in a legend. There may be as many of each symbol as there are plates in the assay.

If assay control samples are not available, a bar chart of endogenous control wells may be provided. The bar for each plate reflects the mean, while the error bars reflect the minimum and maximum C_{T} values. Similar tables and graphs may also be produced for NTC and −RT controls.

In a preferred embodiment of the subject invention, an experiment browser is used as a navigation tool for processing the steps necessary to analyze the experiment. Each step in the process is displayed on the browser. As the user completes a step in the process, that step is marked as complete. This allows the user to determine which steps need to be completed before results can be produced by the calculation step. The experiment browser also provides the following functionality:
Find an existing experiment
Create a new experiment
Delete an experiment
Calculate experiment results
Publish experiment results
Remove experiment results
Navigation for processing steps

Preferably, all users may view all experiments. For example, the experiment owner is often the only user allowed to modify any data for the experiment. It is preferably that certain privileges be established to edit or view a current experiment.

To navigate, the experiments are grouped by year, and month. A folder may be displayed for each year and month for which, based on the find criteria, an experiment exists. Experiments are then preferably ordered by a unique experiment id within each folder. Folders may then be expanded or collapsed. To edit, the appropriate screen is displayed for each step.

In order to work with an experiment in the experiment browser, the desired experiment data must be available is and is preferably retrieved from a remote database. The user may search a database for a particular experiment using experimental criteria or gene criteria or any combination thereof The experiment criteria pertain to a specific experiment. Hence, experiments that match the criteria entered will be retrieved. Example experiment criteria include experiment ID, notebook page, run date and owner. The experiments that match the criteria may be conveniently displayed and viewed below the search criteria.

Gene criteria include criteria pertaining to the species, gene and forward primer, probe, and reverse primer set used in an experiment. Experiments that match the criteria entered will be retrieved and displayed, preferably below the search criteria.

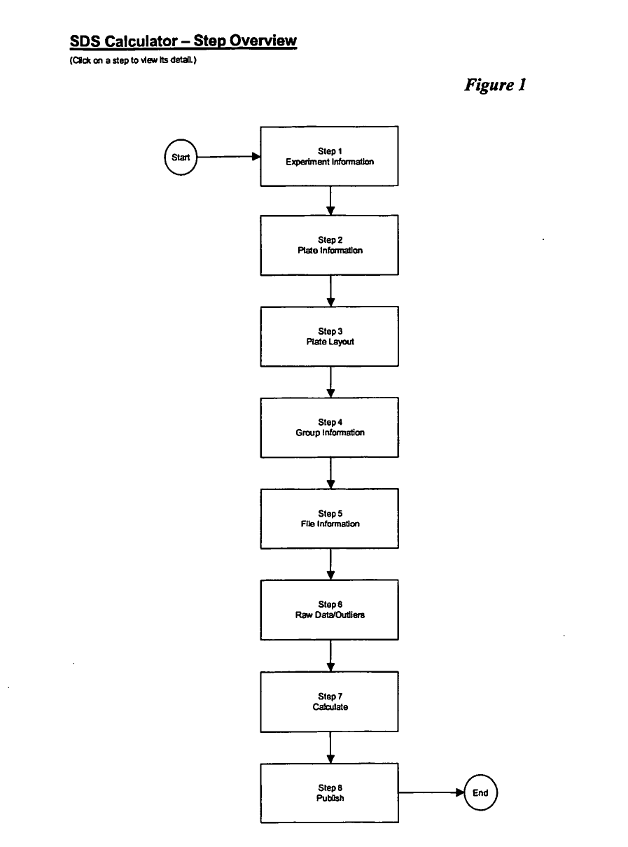

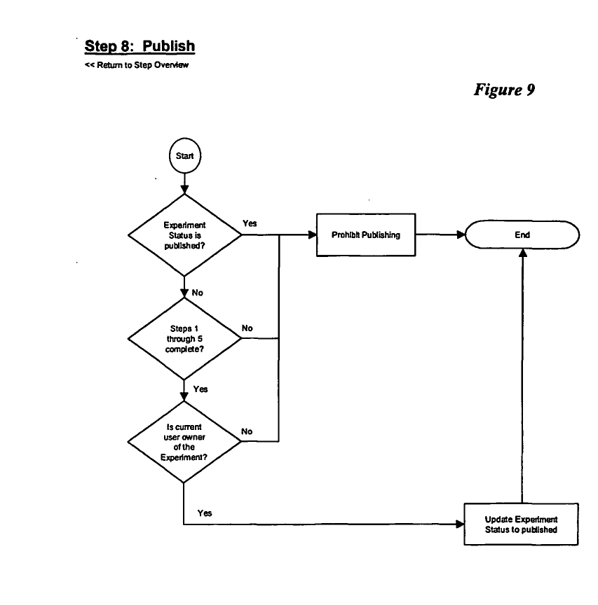

As shown in FIGS. 1 to 9, the method of the subject invention comprises a number of specific steps. FIG. 1 depicts the overall methodology of present invention.

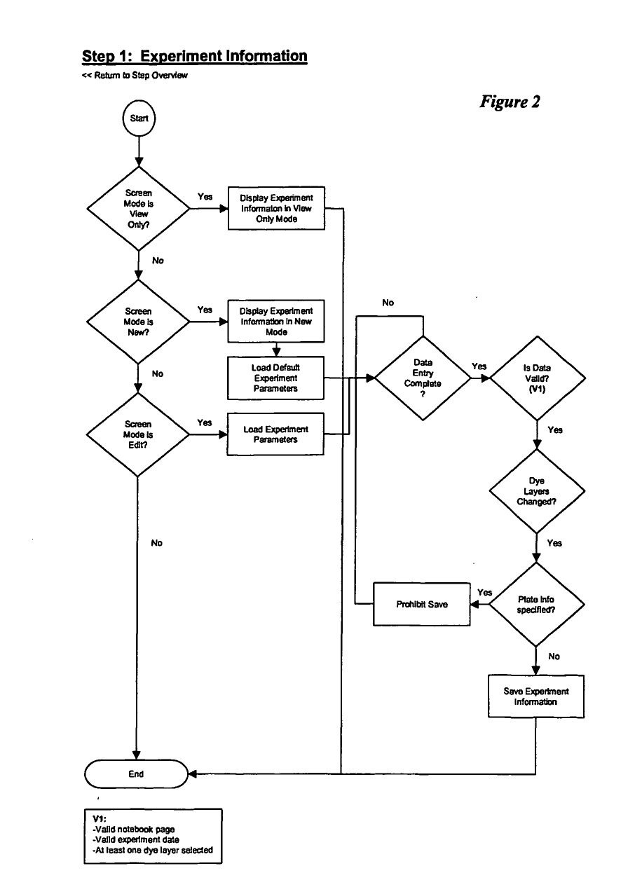

FIG. 2 is a flow chart of the first step, the recording of experiment information. To create a new experiment, a separate screen is displayed and information provided such as experiment ID, description, dye layers and other parameters including notebook page reference, outlier cutoff, and amplification efficiency “E.” Experiments can also be deleted from the database. However, it is recommended that a privilege be attached to this function.

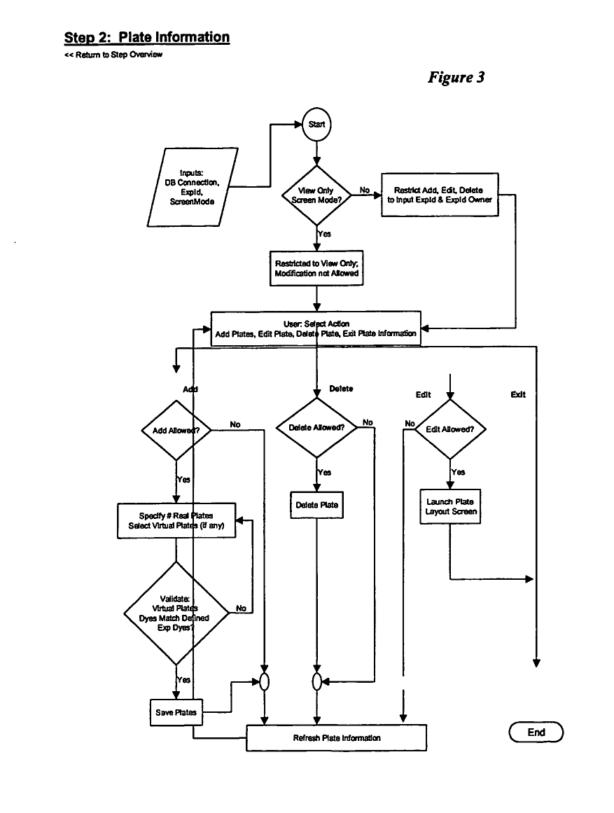

In the step 2, plate information including the number of plates and type of plates are specified. FIG. 3 is a flow chart of this second step. Real or virtual plates may be specified. A virtual plate may be a plate from a previous experiment. Plates of varying size may be selected. For a new experiment there are initially no plates defined. Plates may be added to the experiment in an unlimited number as real or virtual plates.

Real plates are the new plates defined for the current experiment. Data files gathered at the time of the experiment shall be parsed and recorded under the appropriate plate. A type of plate is also chosen such as 96 well or 384 well plate or custom card.

Virtual plates are plates that already exist on another experiment. The data for these plates was gathered on the other experiment. Virtual plates are optional.

For example, the first experiment is at time zero, the second experiment is at time 3 months, and the third and current experiment at time 6 months. The analysis for this current experiment would include the plate date from the previous two experiments, time zero and time 3 months. The current experiment, time 6 months, would then include its own plates (real plates), along with the plates from the previous two experiments, time zero and time 3 months, as virtual plates. When adding virtual plates to an experiment, the dyes used on the virtual plate must match the dyes for the experiment. For example, an experiment defined as using the FAM dye cannot have a virtual plate using the VIC dye.

When specifying plate information, information about the particular plate is included such as number of wells, well type, dye layers, and FPR set. The contents of the well or well type may be minus RT, plate consistency control, sample, and sample and plate consistency control. Each well either contains RNA or is NTC. All wells that are not empty contain an FPR set.

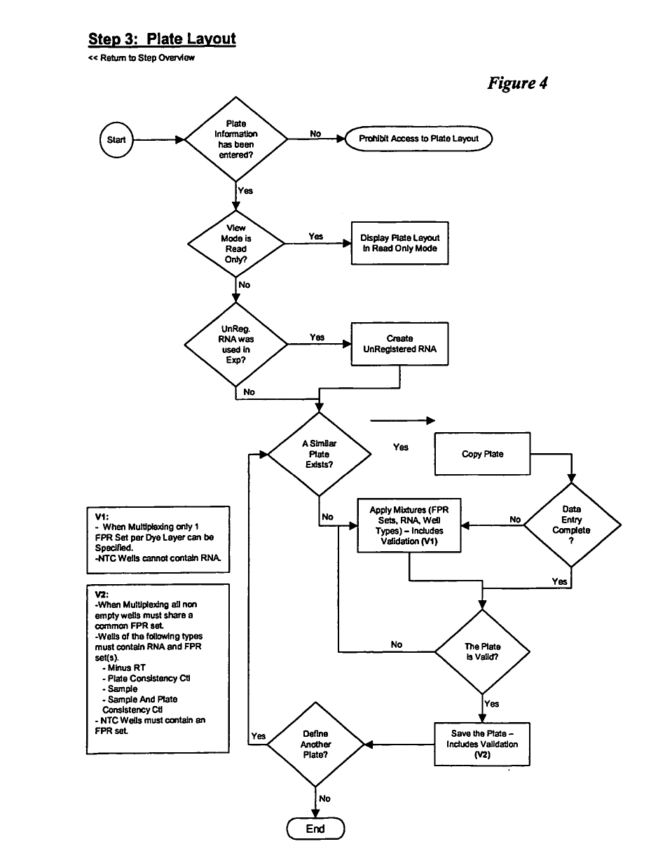

In Step 3, and as shown in FIG. 4, the plate layout including defining FPR sets and RNA associated to each well on the plate for the experiment is provided. Prior to generating this information, both experiment information and plate information must have been completed. The FPR sets are categorized by dye layer and species. To apply an FPR set, select the wells of interest and select the desired FPR set. Conversely, the remove an FPR set, select the wells of interest and delete or remove the FPR set from its designation.

If the experiment is multiplexed, only one FPR set per each dye layer may be used in each well. Dye layers are associated to the experiment through the experiment information. If the experiment is not multiplexed, only one FPR set per well can be specified. When applying FPR sets, if any of the selected wells already contain an FPR set they will not be overridden with the FPR set that is currently selected. To replace an FPR set, the existing FPR set must be removed or deleted first.

RNA is categorized by the user and once recorded as part of an internal database is referred to as registered. When the user changes, the relevant registered RNAs are listed. To apply registered RNA, select the wells of interest and the registered RNA. To remove registered RNA, select the wells of interest and delete the registered RNA. Only one registered RNA per well may be specified. Whey applying registered RNA, if any of the selected wells already contain registered RNA, they will be overridden with registered RNA that is currently selected. To replace registered RNA, it must be removed. Registered RNA cannot be applied to NTC wells or empty wells.

To create unregistered RNA or RNA that has not been previously recorded, identify the number of unregistered RNA to generate. At this time, the name, notebook page and comments may be associated to the unregistered RNA. This unregistered RNA information may be modified if necessary. The unregistered RNA is then associated with wells of interest. Only one unregistered RNA may be specified per well. Unregistered RNA will not be applied to wells already containing unregistered RNA. The unregistered RNA must be removed from a well prior to selecting another unregistered RNA. Unregistered RNA may not be applied to NTC wells or empty wells.

A number of various well types are available for use in connection with the method of the subject invention. The types of wells include, but are not limited to, the following: sample, NTC, RT, plate consistency, sample and plate consistency, or empty. Plate information including FPR sets, registered RNA, unregistered RNA, and well type may be copied from another plate. In order to save plate information, wells of the following types must contain RNA and FPR sets: Minus RT, Plate consistency control, sample, and sample and plate consistency control. NTC wells must contain an FPR set. When multiplexing, all non-empty wells must share a common FPR set.

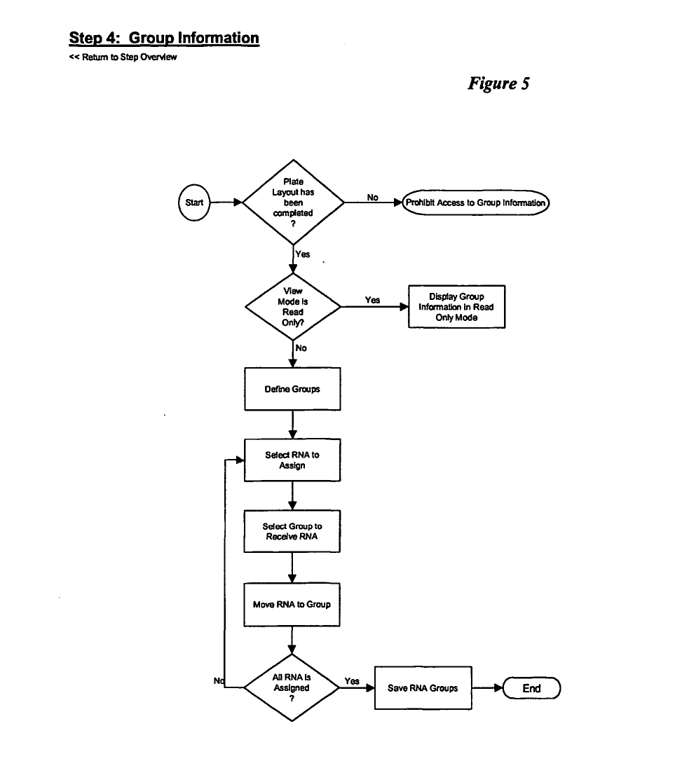

The next step (step 4) in the method of the subject invention is to create and populate RNA groups. FIG. 5 is a flow chart of this step. An RNA group can be only one RNA but may contain multiple RNAs. Both registered and unregistered RNA are available to assign to groups. Only RNA belonging to a sample or sample and plate consistency wells is provided here. Each new RNA group shall have a group name. Each specific RNA is assigned to a group and may be later removed if necessary. All RNA must be assigned to at least one group.

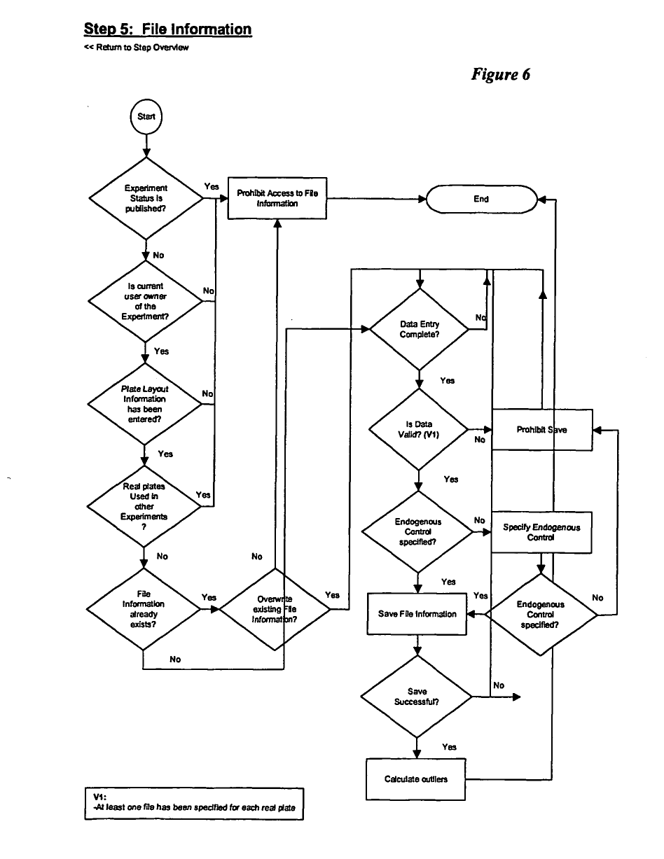

In step 5, exported data files are associated to specific real plates in the experiment. As shown in FIG. 6, the file information for virtual plates used in the experiment already exists and may be overwritten. Any one of a number of data file formats may be utilized. If an endogenous control was not specified, an endogenous control gene must be selected at this time.

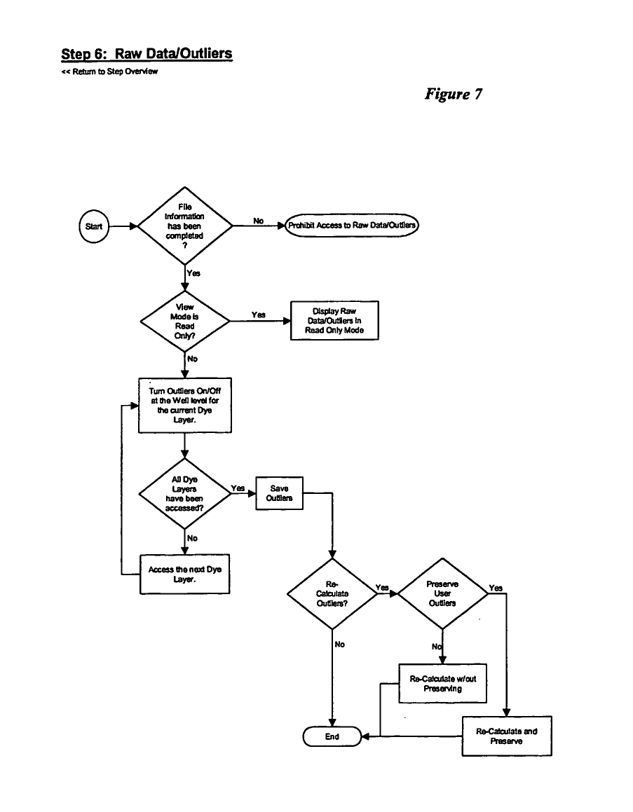

In step 6, C_{T} values may be reviewed and outliers managed. Outliers may be calculated at any point, up to the time the experiment has been published. As shown in FIG. 7, outliers may be turned on or off at the well level for each dye layer. Two types of outliers exist including auto outliers identified during the file information step and user outliers explicitly set by the user. Several outlier values may be identified at one time. When multiplexing, outliers may be viewed for different dye layers. Once all dye layers have been accessed, outliers may be saved or recalculated.

Outliers are determined by calculating the coefficient of variation, CV, for each set of replicate Ct values within the same RNA Group. A replicate Ct value is defined as a sample well containing the same FPR Set and the same RNA. When multiplexing, a sample well may contain multiple Ct values. If the CV for a replicate Ct value exceeds a predetermined percentage, that Ct value is marked as a auto outlier. Marking a Ct value as an auto outlier indicates that the user should review that Ct value for accuracy. If the user determines that the Ct value should not be included in any calculations, the user has the ability to mark it as a user outlier. Marking a Ct value as a user outlier prevents that value from being used in any calculations.

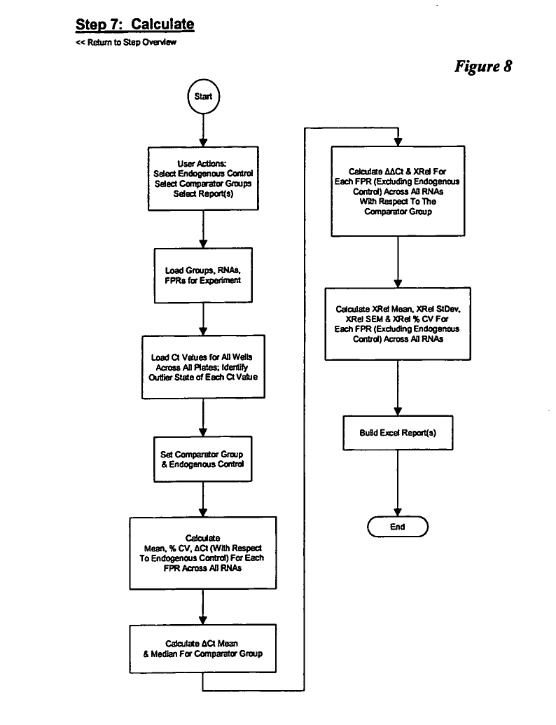

In step 7, as shown in FIG. 8, the calculations are completed. First, the endogenous control and comparative groups are selected. The endogenous control and comparative groups are the basis behind the reported calculation for all genes. Choosing different comparative groups is a unique feature of the method of the subject invention. Through this feature it is possible to compare delta delta C_{T} and XRel results with different comparative groups. The user may exclude marked outliers if necessary.

The endogenous control is initially selected by the user at the time data are parsed for the experiment (step 5 described above). The auto outlier process is performed any time data are changed in experiment analysis. The user may select a different endogenous control during the calculations (step 7) of the analysis. If the endogenous control is changed, the user may run the outlier process again to reflect a change in the endogenous control.

In order to determine the relative expression value of any given sample, one sample (RNA) or group of samples (group of RNAs) must be chosen as a comparator. The comparator group is one to which all other groups will be compared. All relative expression values are defined relative to the comparator group as being the same, higher or lower than the comparator group.

Occasionally in an experiment, there may be a need to calculate relative expression values several times using more than one comparator group. For example, it may be necessary to see relative fold changes in a message compared to different points in the experiment thus creating the need to easily be able to change the comparator group and quickly recalculate the relative expression values.

The ability to chose different comparator groups is a feature of the subject invention that makes it possible to compare ΔΔC_{T} and XREL results using different comparator groups.

The following calculations are made with respect to each endogenous control for each FPR set across all RNAs: mean, % CV and delta C_{T}. Calculations for each comparator group include delta C_{T} mean and median. Across all RNAs with respect to the comparator group the delta delta C_{T} and XRel for each FPR set is calculated. XRel Mean, XRel standard deviation, XRel SEM and XRel % CV is calculated for each FPR set across all RNAs excluding endogenous control.

### EXAMPLE 1

### Experimental Analysis of the Expression of Four Genes in Three Groups

A gene expression experiment was performed analyzing the effects of two different experimental conditions (Groups A and B) relative to a control (Group V) on the expression of five different genes (Genes 1-5). RNA was isolated from seven replicates for the control and nine replicates for the experimental conditions. After isolation, samples are subjected to reverse transcriptase PCR analysis. Each sample was amplified with an endogenous control FPR as well as the FPR's for each of the five genes. Note, this example is not multiplex; multiplex has more than one set of primers and probes in the same reaction well with each probe labeled with a different fluorescent reporter dye. The analysis was performed in duplicate for each sample, requiring a total of four plates to perform all amplifications.

The analysis was initiated for five different genes under three separate experimental conditions. Each experimental condition represents a group, which are encoded herein as Groups A, B and V. The genes are encoded herein as Gene 1, Gene 2, Gene 3, Gene 4 and Gene 5. The C_{T} value for each well were exported. The exported data is shown below in Table 1. Table 1 contains the Ct values extracted from the data files in a format that represents the location of each Ct value on the plate. An EXCEL worksheet may be created for each dye layer used in the experiment. The worksheet contains the Ct values for each plate of the specified dye layer. The C_{T} value for each well is shown relative to the position of the well on the plate. Similar calculations are used to calculate ΔC_{T} values for all of the samples in Groups A (Table 3) and Group B (Table 4). A summary of results calculated from an analysis of all five genes is provided in Table 5.

TABLE 1

| Plate 1 | | | test 2a | | | | | | | | |
|---|---|---|---|---|---|---|---|---|---|---|---|
| 15.83 | 16.03 | 27.06 | 27.18 | 23.35 | 23.19 | 20.57 | 19.96 | 23.82 | 23.79 | 29.09 | 29.36 |
| 16.86 | 16.47 | 26.86 | 27.04 | 23.35 | 23.72 | 21 | 20.64 | 22.78 | 22.3 | 28.78 | 28.43 |
| 16.3 | 16.08 | 27.18 | 27.41 | 23.47 | 23.49 | 20.46 | 20.43 | 23.58 | 23.33 | 28.69 | 29.1 |
| 16.22 | 16.53 | 27.2 | 27.5 | 23.54 | 23.98 | 21.3 | 21.02 | 23.98 | 23.94 | 29.35 | 29.34 |
| 16.23 | 15.96 | 26.64 | 26.9 | 23.73 | 23.94 | 20.53 | 20.61 | 24.6 | 24.31 | 29.08 | 29.01 |
| 16.34 | 17.14 | 26.16 | 26.53 | 23.67 | 23.42 | 21.2 | 21.37 | 25.22 | 25.25 | 29.03 | 29.1 |
| 16.46 | 16.63 | 25.16 | 24.62 | 23.08 | 23.04 | 21.11 | 20.87 | 22.52 | 23.18 | 29.08 | 28.34 |
| 40 | 40 | 40 | 40 | 40 | 40 | 40 | 40 | 40 | 40 | 40 | 40 |
| Plate 2 | | | Plate 371 | | | | | | | | |
| 15.78 | 16.46 | 27.28 | 27.11 | 23.14 | 23.46 | 20.1 | 20.29 | 23.24 | 23.64 | 28.08 | 28.47 |
| 15.82 | 16.25 | 23.04 | 23.32 | 18.89 | 18.42 | 18.87 | 18.43 | 22.25 | 21.85 | 26.06 | 25.87 |
| 16.22 | 16.12 | 23.83 | 24.18 | 19.33 | 18.85 | 19.16 | 18.77 | 22.22 | 22.33 | 26.61 | 26.55 |
| 15.45 | 15.99 | 22.84 | 23.25 | 18.62 | 18.46 | 18.28 | 18.94 | 22.09 | 21.71 | 25.28 | 24.73 |
| 15.99 | 15.57 | 23.49 | 23.44 | 18.94 | 19.22 | 18.21 | 18.44 | 22.33 | 22.76 | 25.17 | 25.02 |
| 15.53 | 16.07 | 24.99 | 24.55 | 19.47 | 19.54 | 19.47 | 19.64 | 22.83 | 23.34 | 27.04 | 27.55 |
| 16.23 | 16.33 | 23.27 | 23.11 | 19.56 | 19.24 | 19.25 | 19.26 | 22 | 22.12 | 26.97 | 26.66 |
| 40 | 40 | 30.13 | 40 | 40 | 40 | 40 | 40 | 40 | 40 | 40 | 40 |
| Plate 3 | | | Plate 372 | | | | | | | | |
| 16.67 | 16.64 | 27.42 | 28.07 | 23.19 | 23.74 | 21.03 | 21.29 | 23.4 | 23.81 | 29.15 | 29.21 |
| 16.55 | 16.4 | 24.5 | 24.68 | 19.04 | 19.25 | 20.31 | 19.68 | 23.37 | 23.24 | 27.21 | 27.2 |
| 16.23 | 15.85 | 24.69 | 25.27 | 19.31 | 19.47 | 19.14 | 19.95 | 23.12 | 22.83 | 26.48 | 26.49 |
| 16.65 | 16.34 | 25.44 | 25.7 | 19.03 | 18.56 | 21.59 | 21.99 | 24.98 | 24.91 | 28.22 | 27.72 |
| 16.43 | 16.15 | 26.57 | 26.61 | 23.03 | 22.65 | 19.71 | 20.2 | 24.07 | 23.7 | 27.19 | 27.78 |
| 16.59 | 16.29 | 26.3 | 26.24 | 22.84 | 23.17 | 21.49 | 21.23 | 24.35 | 24.2 | 28.42 | 28.83 |
| 15.82 | 16.03 | 23.35 | 23.29 | 19.48 | 19.3 | 19.13 | 18.89 | 22.06 | 21.9 | 25.05 | 24.99 |
| 40 | 40 | 40 | 40 | 40 | 40 | 40 | 40 | 40 | 40 | 40 | 40 |
| Plate 4 | | | Plate 373 | | | | | | | | |
| 16.31 | 16.08 | 26.97 | 26.52 | 23.4 | 23.28 | 20.56 | 20.22 | 23.11 | 23.21 | 28.41 | 28.36 |
| 16.09 | 16.17 | 25.77 | 25.88 | 22.26 | 22.42 | 20.32 | 20.21 | 23.11 | 23.34 | 27.72 | 27.85 |
| 16.35 | 16.81 | 24.77 | 24.81 | 21.48 | 21.51 | 19.81 | 20.16 | 22.66 | 22.86 | 27.69 | 28 |
| 16.19 | 15.76 | 25.42 | 25.22 | 21.97 | 21.67 | 19.37 | 19.47 | 23.28 | 22.93 | 27.26 | 27.09 |
| 16.39 | 16.73 | 26.22 | 26.04 | 21.02 | 21.4 | 19.3 | 19.56 | 23.38 | 23.31 | 27.31 | 27.15 |
| 16.55 | 15.98 | 25.87 | 25.79 | 23.08 | 22.99 | 20.49 | 20.55 | 22.78 | 23.17 | 27.15 | 27.51 |
| 16.06 | 16.31 | 25.76 | 25.64 | 22.05 | 21.71 | 20.68 | 20.27 | 23.88 | 23.74 | 27.99 | 28.53 |
| 40 | 40 | 40 | 40 | 40 | 40 | 40 | 40 | 40 | 40 | 40 | 40 |

TABLE 2

| | | | | | | | | | | | | | | Relative | | |
|---|---|---|---|---|---|---|---|---|---|---|---|---|---|---|---|---|
| Sample | Group | Endo CT | Avg CT | % CV | A* | U** | CT | Avg CT | % CV | A* | U** | ΔCT | ΔΔCT | Quantitative | | |
| | | 15.83 | | | | | 23.35 | | | | | | | | n | 7 |
| 1 | V | 16.03 | 15.93 | 0.9 | | | 23.19 | 23.27 | 0.5 | | | 7.34 | 0.05 | 0.97 | MEAN | 1.15 |
| | | 16.47 | | | | | 23.72 | | | | | | | | STDEV | 0.34 |
| 4 | V | 16.86 | 16.67 | 1.7 | | | 23.35 | 23.54 | 1.1 | | | 6.87 | −0.42 | 1.34 | SEM | 0.13 |
| | | 16.08 | | | | | 23.49 | | | | | | | | | |
| 5 | V | 16.30 | 16.19 | 1.0 | | | 23.47 | 23.48 | 0.1 | | | 7.29 | 0.00 | 1.00 | | |
| | | 16.53 | | | | | 23.98 | | | | | | | | | |
| 6 | V | 16.22 | 16.38 | 1.3 | | | 23.54 | 23.76 | 1.3 | | | 7.39 | 0.09 | 0.94 | | |
| | | 15.96 | | | | | 23.94 | | | | | | | | | |
| 7 | V | 16.23 | 16.10 | 1.2 | | | 23.73 | 23.84 | 0.6 | | | 7.74 | 0.45 | 0.73 | | |
| | | 17.14 | | | X | | 23.42 | | | | | | | | | |
| 8 | V | 16.34 | 16.74 | 3.4 | X | | 23.67 | 23.55 | 0.8 | | | 6.81 | −0.48 | 1.40 | | |
| | | 16.63 | | | | | 23.04 | | | | | | | | | |
| 9 | V | 16.46 | 16.55 | 0.7 | | | 23.08 | 23.06 | 0.1 | | | 6.52 | −0.78 | 1.71 | | |

| | |
|---|---|
| MEDIAN ΔCT Vehicle | 7.29 |
| MEAN ΔCT Vehicle | 7.14 |

TABLE 3

| | | | | | | | | | | | | | | Relative | | |
|---|---|---|---|---|---|---|---|---|---|---|---|---|---|---|---|---|
| Sample | Group | Endo CT | Avg CT | % CV | A* | U** | CT | Avg CT | % CV | A* | U** | ΔCT | ΔΔCT | Quantitative | | |
| | | 16.25 | | | | | 18.42 | | | | | | | | n | 9 |
| 10 | A | 15.82 | 16.04 | 1.9 | | | 18.89 | 18.66 | 1.8 | | | 2.62 | −4.67 | 25.46 | MEAN | 20.66 |
| | | 16.12 | | | | | 18.85 | | | | | | | | STDEV | 6.10 |
| 11 | A | 16.22 | 16.17 | 0.4 | | | 19.33 | 19.09 | 1.8 | | | 2.92 | −4.37 | 20.68 | SEM | 2.03 |
| | | 15.99 | | | | | 18.62 | | | | | | | | | |
| 12 | A | 15.45 | 15.72 | 2.4 | | | 18.46 | 18.54 | 0.6 | | | 2.82 | −4.47 | 22.16 | | |
| | | 15.57 | | | | | 19.22 | | | | | | | | | |
| 13 | A | 15.99 | 15.78 | 1.9 | | | 18.94 | 19.08 | 1.0 | | | 3.30 | −3.99 | 15.89 | | |
| | | 15.53 | | | | | 19.54 | | | | | | | | | |
| 14 | A | 16.07 | 15.80 | 2.4 | | | 19.47 | 19.51 | 0.3 | | | 3.71 | −3.59 | 12.00 | | |
| | | 16.33 | | | | | 19.24 | | | | | | | | | |
| 15 | A | 16.23 | 16.28 | 0.4 | | | 19.56 | 19.40 | 1.2 | | | 3.12 | −4.17 | 18.00 | | |
| | | 16.40 | | | | | 19.25 | | | | | | | | | |
| 17 | A | 16.55 | 16.48 | 0.6 | | | 19.04 | 19.15 | 0.8 | | | 2.67 | −4.62 | 24.59 | | |
| | | 15.85 | | | | | 19.47 | | | | | | | | | |
| 18 | A | 16.23 | 16.04 | 1.7 | | | 19.31 | 19.39 | 0.6 | | | 3.35 | −3.94 | 15.35 | | |
| | | 16.34 | | | | | 18.56 | | | | | | | | | |
| 36 | A | 16.65 | 16.50 | 1.3 | | | 19.03 | 18.80 | 1.8 | | | 2.30 | −4.99 | 31.78 | | |

TABLE 4

| | | | | | | | | | | | | | | Relative | | |
|---|---|---|---|---|---|---|---|---|---|---|---|---|---|---|---|---|
| Sample | Group | Endo CT | Avg CT | % CV | A* | U** | CT | Avg CT | % CV | A* | U** | ΔCT | ΔΔCT | Quantitative | | |
| | | 16.15 | | | | | 22.65 | | | | | | | | n | 9 |
| 39 | B | 16.43 | 16.29 | 1.2 | | | 23.03 | 22.84 | 1.2 | | | 6.55 | −0.74 | 1.67 | MEAN | 4.25 |
| | | 16.29 | | | | | 23.17 | | | | | | | | STDEV | 4.08 |
| 40 | B | 16.59 | 16.44 | 1.3 | | | 22.84 | 23.01 | 1.0 | | | 6.57 | −0.72 | 1.65 | SEM | 1.36 |
| | | 16.03 | | | | | 19.30 | | | | | | | | | |
| 41 | B | 15.82 | 15.93 | 0.9 | | | 19.48 | 19.39 | 0.7 | | | 3.47 | −3.83 | 14.17 | | |
| | | 16.17 | | | | | 22.42 | | | | | | | | | |
| 42 | B | 16.09 | 16.13 | 0.4 | | | 22.26 | 22.34 | 0.5 | | | 6.21 | −1.08 | 2.11 | | |
| | | 16.81 | | | | | 21.51 | | | | | | | | | |
| 43 | B | 16.35 | 16.58 | 2.0 | | | 21.48 | 21.50 | 0.1 | | | 4.92 | −2.38 | 5.19 | | |
| | | 15.76 | | | | | 21.67 | | | | | | | | | |
| 44 | B | 16.19 | 15.98 | 1.9 | | | 21.97 | 21.82 | 1.0 | | | 5.85 | −1.45 | 2.72 | | |
| | | 16.73 | | | | | 21.40 | | | | | | | | | |
| 46 | B | 16.39 | 16.56 | 1.5 | | | 21.02 | 21.21 | 1.3 | | | 4.65 | −2.64 | 6.23 | | |
| | | 15.98 | | | | | 22.99 | | | | | | | | | |
| 47 | B | 16.55 | 16.27 | 2.5 | | | 23.08 | 23.04 | 0.3 | | | 6.77 | −0.52 | 1.43 | | |
| | | 16.31 | | | | | 21.71 | | | | | | | | | |
| 48 | B | 16.06 | 16.19 | 1.1 | | | 22.05 | 21.88 | 1.1 | | | 5.70 | −1.60 | 3.02 | | |

TABLE 5

| | | | |
|---|---|---|---|
| Experiment ID: | 99999 | Description: | Experiment |
| Description | | | |
| Label: | Experiment Label | | |
| Experiment Date: | 8/21/2001 | | |
| Amplification Efficiency (E): | 1 | | |
| Outlier Cutoff (% CV): | 3 | | |

| | | | | | | | | Relative |
|---|---|---|---|---|---|---|---|---|
| Dye | Group | Sample | Gene | Avg CT | % CV | ΔCT | ΔΔCT | Quantitative SEM |
| Dye Layer 1 | V | 1 | Endo | 15.93 | 0.9 | N/A | N/A | N/A |
| Dye Layer 1 | V | 1 | Gene 1 | 23.27 | 0.5 | 7.34 | 0.05 | 0.97 |
| Dye Layer 1 | V | 1 | Gene 2 | 20.27 | 2.1 | 4.34 | −0.11 | 1.08 |
| Dye Layer 1 | V | 1 | Gene 3 | 23.81 | 0.1 | 7.88 | 0.29 | 0.82 |
| Dye Layer 1 | V | 1 | Gene 4 | 29.23 | 0.7 | 13.30 | 0.59 | 0.66 |
| Dye Layer 1 | V | 1 | Gene 5 | 27.12 | 0.3 | 11.19 | 0.51 | 0.70 |
| Dye Layer 1 | V | 4 | Endo | 16.67 | 1.7 | N/A | N/A | N/A |
| Dye Layer 1 | V | 4 | Gene 1 | 23.54 | 1.1 | 6.87 | −0.42 | 1.34 |
| Dye Layer 1 | V | 4 | Gene 2 | 20.82 | 1.2 | 4.16 | −0.29 | 1.22 |
| Dye Layer 1 | V | 4 | Gene 3 | 22.54 | 1.5 | 5.88 | −1.71 | 3.27 |
| Dye Layer 1 | V | 4 | Gene 4 | 28.61 | 0.9 | 11.94 | −0.77 | 1.70 |
| Dye Layer 1 | V | 4 | Gene 5 | 26.95 | 0.5 | 10.29 | −0.39 | 1.31 |
| Dye Layer 1 | V | 5 | Endo | 16.19 | 1.0 | N/A | N/A | N/A |
| Dye Layer 1 | V | 5 | Gene 1 | 23.48 | 0.1 | 7.29 | 0.00 | 1.00 |
| Dye Layer 1 | V | 5 | Gene 2 | 20.45 | 0.1 | 4.26 | −0.19 | 1.14 |
| Dye Layer 1 | V | 5 | Gene 3 | 23.46 | 0.8 | 7.27 | −0.32 | 1.25 |
| Dye Layer 1 | V | 5 | Gene 4 | 28.90 | 1.0 | 12.71 | 0.00 | 1.00 |
| Dye Layer 1 | V | 5 | Gene 5 | 27.30 | 0.6 | 11.11 | 0.43 | 0.74 |
| Dye Layer 1 | V | 6 | Endo | 16.38 | 1.3 | N/A | N/A | N/A |
| Dye Layer 1 | V | 6 | Gene 1 | 23.76 | 1.3 | 7.39 | 0.09 | 0.94 |
| Dye Layer 1 | V | 6 | Gene 2 | 21.16 | 0.9 | 4.79 | 0.34 | 0.79 |
| Dye Layer 1 | V | 6 | Gene 3 | 23.96 | 0.1 | 7.59 | 0.00 | 1.00 |
| Dye Layer 1 | V | 6 | Gene 4 | 29.35 | 0.0 | 12.97 | 0.26 | 0.83 |
| Dye Layer 1 | V | 6 | Gene 5 | 27.35 | 0.8 | 10.98 | 0.30 | 0.81 |
| Dye Layer 1 | V | 7 | Endo | 16.10 | 1.2 | N/A | N/A | N/A |
| Dye Layer 1 | V | 7 | Gene 1 | 23.84 | 0.6 | 7.74 | 0.45 | 0.73 |
| Dye Layer 1 | V | 7 | Gene 2 | 20.57 | 0.3 | 4.48 | 0.03 | 0.98 |
| Dye Layer 1 | V | 7 | Gene 3 | 24.46 | 0.8 | 8.36 | 0.77 | 0.58 |
| Dye Layer 1 | V | 7 | Gene 4 | 29.05 | 0.2 | 12.95 | 0.24 | 0.84 |
| Dye Layer 1 | V | 7 | Gene 5 | 26.77 | 0.7 | 10.68 | 0.00 | 1.00 |
| Dye Layer 1 | V | 8 | Endo | 16.74 | 3.4 | N/A | N/A | N/A |
| Dye Layer 1 | V | 8 | Gene 1 | 23.55 | 0.8 | 6.81 | −0.48 | 1.40 |
| Dye Layer 1 | V | 8 | Gene 2 | 21.29 | 0.6 | 4.55 | 0.10 | 0.93 |
| Dye Layer 1 | V | 8 | Gene 3 | 25.24 | 0.1 | 8.50 | 0.91 | 0.53 |
| Dye Layer 1 | V | 8 | Gene 4 | 29.07 | 0.2 | 12.33 | −0.38 | 1.30 |
| Dye Layer 1 | V | 8 | Gene 5 | 26.35 | 1.0 | 9.61 | −1.07 | 2.10 |
| Dye Layer 1 | V | 9 | Endo | 16.55 | 0.7 | N/A | N/A | N/A |
| Dye Layer 1 | V | 9 | Gene 1 | 23.06 | 0.1 | 6.52 | −0.78 | 1.71 |
| Dye Layer 1 | V | 9 | Gene 2 | 20.99 | 0.8 | 4.45 | 0.00 | 1.00 |
| Dye Layer 1 | V | 9 | Gene 3 | 22.85 | 2.0 | 6.31 | −1.28 | 2.43 |
| Dye Layer 1 | V | 9 | Gene 4 | 28.71 | 1.8 | 12.17 | −0.54 | 1.45 |
| Dye Layer 1 | V | 9 | Gene 5 | 24.89 | 1.5 | 8.35 | −2.33 | 5.03 |
| Dye Layer 1 | A | 10 | Endo | 16.04 | 1.9 | N/A | N/A | N/A |
| Dye Layer 1 | A | 10 | Gene 1 | 18.66 | 1.8 | 2.62 | −4.67 | 25.46 |
| Dye Layer 1 | A | 10 | Gene 2 | 18.65 | 1.7 | 2.62 | −1.83 | 3.56 |
| Dye Layer 1 | A | 10 | Gene 3 | 22.05 | 1.3 | 6.02 | −1.57 | 2.97 |
| Dye Layer 1 | A | 10 | Gene 4 | 25.97 | 0.5 | 9.93 | −2.78 | 6.84 |
| Dye Layer 1 | A | 10 | Gene 5 | 23.18 | 0.9 | 7.15 | −3.53 | 11.55 |
| Dye Layer 1 | A | 11 | Endo | 16.17 | 0.4 | N/A | N/A | N/A |
| Dye Layer 1 | A | 11 | Gene 1 | 19.09 | 1.8 | 2.92 | −4.37 | 20.68 |
| Dye Layer 1 | A | 11 | Gene 2 | 18.97 | 1.5 | 2.80 | −1.65 | 3.14 |
| Dye Layer 1 | A | 11 | Gene 3 | 22.28 | 0.3 | 6.11 | −1.48 | 2.79 |
| Dye Layer 1 | A | 11 | Gene 4 | 26.58 | 0.2 | 10.41 | −2.30 | 4.91 |
| Dye Layer 1 | A | 11 | Gene 5 | 24.01 | 1.0 | 7.84 | −2.84 | 7.16 |
| Dye Layer 1 | A | 12 | Endo | 15.72 | 2.4 | N/A | N/A | N/A |
| Dye Layer 1 | A | 12 | Gene 1 | 18.54 | 0.6 | 2.82 | −4.47 | 22.16 |
| Dye Layer 1 | A | 12 | Gene 2 | 18.61 | 2.5 | 2.89 | −1.56 | 2.94 |
| Dye Layer 1 | A | 12 | Gene 3 | 21.90 | 1.2 | 6.18 | −1.41 | 2.65 |
| Dye Layer 1 | A | 12 | Gene 4 | 25.01 | 1.6 | 9.29 | −3.42 | 10.70 |
| Dye Layer 1 | A | 12 | Gene 5 | 23.05 | 1.3 | 7.33 | −3.35 | 10.20 |
| Dye Layer 1 | A | 13 | Endo | 15.78 | 1.9 | N/A | N/A | N/A |
| Dye Layer 1 | A | 13 | Gene 1 | 19.08 | 1.0 | 3.30 | −3.99 | 15.89 |
| Dye Layer 1 | A | 13 | Gene 2 | 18.33 | 0.9 | 2.55 | −1.90 | 3.73 |
| Dye Layer 1 | A | 13 | Gene 3 | 22.55 | 1.3 | 6.77 | −0.82 | 1.77 |
| Dye Layer 1 | A | 13 | Gene 4 | 25.10 | 0.4 | 9.32 | −3.39 | 10.48 |
| Dye Layer 1 | A | 13 | Gene 5 | 23.47 | 0.2 | 7.69 | −2.99 | 7.94 |
| Dye Layer 1 | A | 14 | Endo | 15.80 | 2.4 | N/A | N/A | N/A |
| Dye Layer 1 | A | 14 | Gene 1 | 19.51 | 0.3 | 3.71 | −3.59 | 12.00 |
| Dye Layer 1 | A | 14 | Gene 2 | 19.56 | 0.6 | 3.76 | −0.69 | 1.61 |
| Dye Layer 1 | A | 14 | Gene 3 | 23.09 | 1.6 | 7.29 | −0.30 | 1.23 |
| Dye Layer 1 | A | 14 | Gene 4 | 27.30 | 1.3 | 11.50 | −1.21 | 2.31 |
| Dye Layer 1 | A | 14 | Gene 5 | 24.77 | 1.3 | 8.97 | −1.71 | 3.26 |
| Dye Layer 1 | A | 15 | Endo | 16.28 | 0.4 | N/A | N/A | N/A |
| Dye Layer 1 | A | 15 | Gene 1 | 19.40 | 1.2 | 3.12 | −4.17 | 18.00 |
| Dye Layer 1 | A | 15 | Gene 2 | 19.26 | 0.0 | 2.98 | −1.47 | 2.77 |
| Dye Layer 1 | A | 15 | Gene 3 | 22.06 | 0.4 | 5.78 | −1.81 | 3.49 |
| Dye Layer 1 | A | 15 | Gene 4 | 26.82 | 0.8 | 10.54 | −2.17 | 4.50 |
| Dye Layer 1 | A | 15 | Gene 5 | 23.19 | 0.5 | 6.91 | −3.77 | 13.59 |
| Dye Layer 1 | A | 17 | Endo | 16.48 | 0.6 | N/A | N/A | N/A |
| Dye Layer 1 | A | 17 | Gene 1 | 19.15 | 0.8 | 2.67 | −4.62 | 24.59 |
| Dye Layer 1 | A | 17 | Gene 2 | 20.00 | 2.2 | 3.52 | −0.93 | 1.90 |
| Dye Layer 1 | A | 17 | Gene 3 | 23.31 | 0.4 | 6.83 | −0.76 | 1.69 |
| Dye Layer 1 | A | 17 | Gene 4 | 27.21 | 0.0 | 10.73 | −1.98 | 3.93 |
| Dye Layer 1 | A | 17 | Gene 5 | 24.59 | 0.5 | 8.12 | −2.56 | 5.90 |
| Dye Layer 1 | A | 18 | Endo | 16.04 | 1.7 | N/A | N/A | N/A |
| Dye Layer 1 | A | 18 | Gene 1 | 19.39 | 0.6 | 3.35 | −3.94 | 15.35 |
| Dye Layer 1 | A | 18 | Gene 2 | 19.55 | 2.9 | 3.51 | −0.94 | 1.92 |
| Dye Layer 1 | A | 18 | Gene 3 | 22.98 | 0.9 | 6.94 | −0.65 | 1.57 |
| Dye Layer 1 | A | 18 | Gene 4 | 26.49 | 0.0 | 10.45 | −2.26 | 4.79 |
| Dye Layer 1 | A | 18 | Gene 5 | 24.98 | 1.6 | 8.94 | −1.74 | 3.33 |
| Dye Layer 1 | A | 36 | Endo | 16.50 | 1.3 | N/A | N/A | N/A |
| Dye Layer 1 | A | 36 | Gene 1 | 18.80 | 1.8 | 2.30 | −4.99 | 31.78 |
| Dye Layer 1 | A | 36 | Gene 2 | 21.79 | 1.3 | 5.30 | 0.85 | 0.55 |
| Dye Layer 1 | A | 36 | Gene 3 | 24.95 | 0.2 | 8.45 | 0.87 | 0.55 |
| Dye Layer 1 | A | 36 | Gene 4 | 27.97 | 1.3 | 11.48 | −1.23 | 2.35 |
| Dye Layer 1 | A | 36 | Gene 5 | 25.57 | 0.7 | 9.08 | −1.60 | 3.03 |
| Dye Layer 1 | B | 39 | Endo | 16.29 | 1.2 | N/A | N/A | N/A |
| Dye Layer 1 | B | 39 | Gene 1 | 22.84 | 1.2 | 6.55 | −0.74 | 1.67 |
| Dye Layer 1 | B | 39 | Gene 2 | 19.96 | 1.7 | 3.67 | −0.78 | 1.72 |
| Dye Layer 1 | B | 39 | Gene 3 | 23.89 | 1.1 | 7.60 | 0.01 | 0.99 |
| Dye Layer 1 | B | 39 | Gene 4 | 27.49 | 1.5 | 11.20 | −1.51 | 2.85 |
| Dye Layer 1 | B | 39 | Gene 5 | 26.59 | 0.1 | 10.30 | −0.38 | 1.30 |
| Dye Layer 1 | B | 40 | Endo | 16.44 | 1.3 | N/A | N/A | N/A |
| Dye Layer 1 | B | 40 | Gene 1 | 23.01 | 1.0 | 6.57 | −0.72 | 1.65 |
| Dye Layer 1 | B | 40 | Gene 2 | 21.36 | 0.9 | 4.92 | 0.48 | 0.72 |
| Dye Layer 1 | B | 40 | Gene 3 | 24.28 | 0.4 | 7.84 | 0.25 | 0.84 |
| Dye Layer 1 | B | 40 | Gene 4 | 28.63 | 1.0 | 12.19 | −0.52 | 1.43 |
| Dye Layer 1 | B | 40 | Gene 5 | 26.27 | 0.2 | 9.83 | −0.84 | 1.80 |
| Dye Layer 1 | B | 41 | Endo | 15.93 | 0.9 | N/A | N/A | N/A |
| Dye Layer 1 | B | 41 | Gene 1 | 19.39 | 0.7 | 3.47 | −3.83 | 14.17 |
| Dye Layer 1 | B | 41 | Gene 2 | 19.01 | 0.9 | 3.09 | −1.36 | 2.57 |
| Dye Layer 1 | B | 41 | Gene 3 | 21.98 | 0.5 | 6.06 | −1.53 | 2.89 |
| Dye Layer 1 | B | 41 | Gene 4 | 25.02 | 0.2 | 9.10 | −3.61 | 12.21 |
| Dye Layer 1 | B | 41 | Gene 5 | 23.32 | 0.2 | 7.40 | −3.28 | 9.71 |
| Dye Layer 1 | B | 42 | Endo | 16.13 | 0.4 | N/A | N/A | N/A |
| Dye Layer 1 | B | 42 | Gene 1 | 22.34 | 0.5 | 6.21 | −1.08 | 2.11 |
| Dye Layer 1 | B | 42 | Gene 2 | 20.27 | 0.4 | 4.14 | −0.31 | 1.24 |
| Dye Layer 1 | B | 42 | Gene 3 | 23.23 | 0.7 | 7.10 | −0.49 | 1.40 |
| Dye Layer 1 | B | 42 | Gene 4 | 27.79 | 0.3 | 11.66 | −1.05 | 2.07 |
| Dye Layer 1 | B | 42 | Gene 5 | 25.83 | 0.3 | 9.70 | −0.98 | 1.97 |
| Dye Layer 1 | B | 43 | Endo | 16.58 | 2.0 | N/A | N/A | N/A |
| Dye Layer 1 | B | 43 | Gene 1 | 21.50 | 0.1 | 4.92 | −2.38 | 5.19 |
| Dye Layer 1 | B | 43 | Gene 2 | 19.99 | 1.2 | 3.41 | −1.04 | 2.06 |
| Dye Layer 1 | B | 43 | Gene 3 | 22.76 | 0.6 | 6.18 | −1.41 | 2.65 |
| Dye Layer 1 | B | 43 | Gene 4 | 27.85 | 0.8 | 11.27 | −1.44 | 2.71 |
| Dye Layer 1 | B | 43 | Gene 5 | 24.79 | 0.1 | 8.21 | −2.47 | 5.52 |
| Dye Layer 1 | B | 44 | Endo | 15.98 | 1.9 | N/A | N/A | N/A |
| Dye Layer 1 | B | 44 | Gene 1 | 21.82 | 1.0 | 5.85 | −1.45 | 2.72 |
| Dye Layer 1 | B | 44 | Gene 2 | 19.42 | 0.4 | 3.45 | −1.00 | 2.00 |
| Dye Layer 1 | B | 44 | Gene 3 | 23.11 | 1.1 | 7.13 | −0.46 | 1.37 |
| Dye Layer 1 | B | 44 | Gene 4 | 27.18 | 0.4 | 11.20 | −1.51 | 2.84 |
| Dye Layer 1 | B | 44 | Gene 5 | 25.32 | 0.6 | 9.35 | −1.33 | 2.51 |
| Dye Layer 1 | B | 46 | Endo | 16.56 | 1.5 | N/A | N/A | N/A |
| Dye Layer 1 | B | 46 | Gene 1 | 21.21 | 1.3 | 4.65 | −2.64 | 6.23 |
| Dye Layer 1 | B | 46 | Gene 2 | 19.43 | 0.9 | 2.87 | −1.58 | 2.98 |
| Dye Layer 1 | B | 46 | Gene 3 | 23.35 | 0.2 | 6.79 | −0.80 | 1.74 |
| Dye Layer 1 | B | 46 | Gene 4 | 27.23 | 0.4 | 10.67 | −2.04 | 4.10 |
| Dye Layer 1 | B | 46 | Gene 5 | 26.13 | 0.5 | 9.57 | −1.11 | 2.15 |
| Dye Layer 1 | B | 47 | Endo | 16.27 | 2.5 | N/A | N/A | N/A |
| Dye Layer 1 | B | 47 | Gene 1 | 23.04 | 0.3 | 6.77 | −0.52 | 1.43 |
| Dye Layer 1 | B | 47 | Gene 2 | 20.52 | 0.2 | 4.26 | −0.19 | 1.14 |
| Dye Layer 1 | B | 47 | Gene 3 | 22.98 | 1.2 | 6.71 | −0.88 | 1.83 |
| Dye Layer 1 | B | 47 | Gene 4 | 27.33 | 0.9 | 11.07 | −1.64 | 3.12 |
| Dye Layer 1 | B | 47 | Gene 5 | 25.83 | 0.2 | 9.57 | −1.11 | 2.16 |
| Dye Layer 1 | B | 48 | Endo | 16.19 | 1.1 | N/A | N/A | N/A |
| Dye Layer 1 | B | 48 | Gene 1 | 21.88 | 1.1 | 5.70 | −1.60 | 3.02 |
| Dye Layer 1 | B | 48 | Gene 2 | 20.48 | 1.4 | 4.29 | −0.15 | 1.11 |
| Dye Layer 1 | B | 48 | Gene 3 | 23.81 | 0.4 | 7.63 | 0.04 | 0.97 |
| Dye Layer 1 | B | 48 | Gene 4 | 28.26 | 1.4 | 12.08 | −0.63 | 1.55 |
| Dye Layer 1 | B | 48 | Gene 5 | 25.70 | 0.3 | 9.52 | −1.16 | 2.23 |

1. A computer-readable medium containing instructions for controlling a computer system in the analysis of an experiment to detect RNA or DNA in a sample, by:
receiving exported cycle threshold values for a plate of wells;
calculating delta C_{T}, delta delta C_{T} and relative transcriptional change for the sample; and
displaying the cycle threshold values, the delta C_{T}, the delta delta C_{T} and the relative transcription change (XRel) of the sample.

2. A method in a computer system for analyzing an experiment to detect RNA or DNA from a two-dimensional plate configuration comprising the steps of:
recording experiment information for the experiment wherein the experiment information comprises an experiment id;
specify at least one plate to the experiment wherein said plate is a real plate or a virtual plate, each said plate comprising a series of wells and dye layers, each said plate having at least one an FPR set, wherein each said FPR set is categorized by dye layer or well;
create and populate at least one RNA group wherein RNA is assigned to the RNA group;
receive exported experimental cycling results for each said plate including a CT value for each FPR set in each said well;
calculate delta C_{T}, delta delta C_{T}, and XRel values for each said sample RNA; and
display the C_{T}, the delta C_{T}, the delta delta Ct, and the XRel values for each said sample RNA to detect RNA.

3. The method of claim 2, further comprising the step of characterizing a plate layout.

4. The method of claim 2, further comprising the step of accessing raw data and managing outliers.

5. The method of claim 4, wherein the outliers are managed at the well level for said each dye layer.

6. The method of claim 2, further comprising the step of preparing an experiment for a sample wherein the experiment is a new experiment or an existing experiment.

7. The method of claim 2, wherein file information is linked to each said real plate.

8. The method claim 2, wherein a plate is linked with each said virtual plate.

9. The method of claim 2, wherein said experiment information includes at least one additional piece of information selected from the group of experiment date, dye layer, label, description, notebook page, outlier cutoff, and amplification efficiency.

10. The method of claim 2, wherein said experimental information includes gene criteria having at least one criteria selected from the group of species, gene, forward primer, probe and reverse primer.

11. The method of claim 2, wherein one FPR set is specified per well.

12. The method of claim 2, wherein the experiment is multiplexed and one FPR set is specified per each dye layer

13. The method of claim 2, wherein one registered RNA is specified per well.

14. The method of claim 2, wherein unregistered RNA is specified per well.

15. The method of claim 2, wherein the contents of the well is selected from the group of minus RT, plate consistency control, sample, and sample and plate consistency control, and each said well contains RNA and the FPR set.

16. The method of claim 2, wherein the well type is NTC and the well contains the FPR set.

17. An information-display apparatus used with a sequence detection system having at least one plate, each plate containing a series of wells and at least one FPR set, each well having at least one dye layer, each dye layer operating independently for detection of a fluorescent emission of an up regulator or a down regulator during the polymerse chain reaction, said apparatus comprising:
receiving means for receiving information from said sequence detection system, wherein the received information includes at least calculated threshold data that represents the detection of RNA of each said dye layer;
calculating means for calculating a delta calculated threshold, a delta delta calculated threshold and a relative transcriptional change for a sample; and
displaying means for displaying the received information and/or the calculated sum.

18. A computer program product for use in connection with an information-display apparatus, said computer program comprising:
a computer usable medium having a computer readable program means embodied in said medium comprising a collection of cycle threshold values for each dye layer of each well of a plate for determining the presence of an RNA or DNA sample, said computer readable program means for causing a computer to calculate and display a delta C_{T}, a delta delta C_{T} and a XRel value.

19. An article of manufacture comprising:
a computer usable medium having computer readable program code embodied therein for determining the presence of RNA or DNA in a sample contained within a dye layer of a well of a plate, the computer readable program means in said article of manufacture comprising:
computer readable program code for causing a computer to effect, with respect to one dye layer, receiving a C_{T} value and storing said C_{T} value in an array of data;
computer readable program code for causing the computer to calculate a delta C_{T}, a delta delta C_{T} and a XRel value for each dye layer, and
a computer readable program code for causing the computer to display the delta C_{T}, the delta delta C_{T} and the XRel values for the sample.

20. A program storage device readable by a computer, tangibly embodying a program of instructions executable by the computer to perform method steps for determining the presence of RNA in a sample, by:
receiving exported cycle threshold values for a plate of wells; and
calculating a delta C_{T}, a delta delta C_{T} and a relative transcriptional change for the sample; and
displaying the cycle threshold values, and the delta C_{T}, the delta delta C_{T} and the XRel values of the sample.

21. A memory for storing data for access by a computer readable program being executed on a computer, comprising:
a data structure stored in said memory, said data structure including information resident in a database used by the computer readable program and including:
experiment information;
plate information including raw data outliers;
plate layout;
RNA group information; and
export file information including C_{T} value.

22. A computer-readable medium containing a data structure for storing a collection of information to determine the presence of RNA or DNA in a sample comprising experiment information, plate information including raw data outliers, plate layout, RNA group information, export file information, and C_{T} values.

23. A method in a computer system for calculating and displaying delta C_{T} values, delta delta C_{T} values, and XRel values in the analysis of an experiment to detect RNA or DNA from a two-dimensional plate configuration containing wells, comprising:
identify a well type;
specify an FPR set for the experiment;
specify an RNA group wherein said RNA group has at least one RNA retrieve a C_{T} value for each well of each plate;
selecting at least one comparator group wherein said comparator group has at least one RNA; and
display reports showing calculated delta C_{T}, delta delta C_{T}, and XRel.

24. The method of claim 23, further comprising the step of select an endogenous control.
